(19) United States
(12) Patent Application Publication
(10) Pub. No.: US 2025/0009396 A1
(43) Pub. Date: Jan. 9, 2025

(54) APPARATUS AND METHOD FOR FUSING A SACROILIAC JOINT

(71) Applicant: VGI Medical, LLC, Largo, FL (US)

(72) Inventor: Tov Inge Vestgaarden, Madeira Beach, FL (US)

(21) Appl. No.: 18/739,209

(22) Filed: Jun. 10, 2024

Related U.S. Application Data
(63) Continuation-in-part of application No. 18/208,963, filed on Jun. 13, 2023, now abandoned.
Application No. 18/208,963 is a continuation of application No. 15/899,577, filed on Feb. 20, 2018, now Pat. No. 11,672,574.
Application No. 15/899,577 is a continuation of application No. 15/195,191, filed on Jun. 28, 2016, now Pat. No. 9,895,176.
Application No. 15/195,191 is a continuation of application No. 14/537,327, filed on Nov. 10, 2014, now Pat. No. 9,375,243.
Application No. 14/537,327 is a continuation of application No. 13/625,180, filed on Sep. 24, 2012, now Pat. No. 8,882,818.
(60) Provisional application No. 63/471,788, filed on Jun. 8, 2023.

(51) Int. Cl. A61B 17/70; A61B 17/02; A61B 17/16; A61B 17/17; A61B 17/56; A61B 17/68; A61B 17/84; A61B 17/88

(52) U.S. Cl. CPC A61B 17/7074; A61B 17/025; A61B 17/1604; A61B 17/1615; A61B 17/1662; A61B 17/1671; A61B 17/1739; A61B 17/68; A61B 17/846; A61B 17/8805; A61B 2017/0256; A61B 2017/564

(57) ABSTRACT

A stabilization implant comprising: an elongated body having a distal end, a proximal end and a longitudinal axis extending between the distal end and the proximal end, the elongated body being characterized by a superior body surface and an inferior body surface; a superior stabilizer extending outwardly from the superior body surface, the superior stabilizer being characterized by a first lateral superior stabilizer surface and a second lateral superior stabilizer surface; and an inferior stabilizer extending outwardly from the inferior body surface, the inferior stabilizer being characterized by a first lateral inferior stabilizer surface and a second lateral inferior stabilizer surface; wherein at least one of the first lateral superior stabilizer surface, the second lateral superior stabilizer surface, the first lateral inferior stabilizer surface, and the second lateral inferior stabilizer surface comprise at least one outwardly-extending tab.

### REFERENCE TO PENDING PRIOR PATENT APPLICATIONS

This patent application:
(i) is a continuation-in-part of pending prior U.S. patent application Ser. No. 18/208,963, filed Jun. 13, 2023 by Tov Inge Vestgaarden and VGI Medical, LLC for METHOD FOR DEPLOYING A FUSION DEVICE FOR SACROILIAC JOINT FUSION (Attorney's Docket No. VG-2425.08 CON 4), which patent application, in turn is a continuation of prior U.S. patent application Ser. No. 15/899,577, filed Feb. 20, 2018 by Tov Inge Vestgaarden and VGI Medical, LLC for METHOD FOR DEPLOYING A FUSION DEVICE FOR SACROILIAC JOINT FUSION (Attorney's Docket No. VG-2425.08 CON 3), which patent application, in turn is a continuation of prior U.S. patent application Ser. No. 15/195,191, filed Jun. 28, 2016 by Tov Inge Vestgaarden and VGI Medical, LLC for METHOD FOR DEPLOYING A FUSION DEVICE FOR SACROILIAC JOINT FUSION (Attorney's Docket No. VG-2425.08 CON 2), which patent application, in turn is a continuation of prior U.S. patent application Ser. No. 14/537,327, filed Nov. 10, 2014 by Tov Inge Vestgaarden and VGI Medical, LLC for METHOD FOR DEPLOYING A FUSION DEVICE FOR SACROILIAC JOINT FUSION (Attorney's Docket No. VG-2425.08 CON), which patent application, in turn (a) is a continuation of prior U.S. patent application Ser. No. 13/625,180, filed Sep. 24, 2012 by Tov Inge Vestgaarden and VGI Medical, LLC for METHOD FOR DEPLOYING A FUSION DEVICE FOR SACROILIAC JOINT FUSION (Attorney's Docket No. VG-2425.08); and
(ii) claims benefit of pending prior U.S. Provisional Patent Application Ser. No. 63/471,788, filed Jun. 8, 2023 by Tov Inge Vestgaarden and VGI Medical, LLC for ALTERNATIVE STABILIZATION IMPLANT (Attorney's Docket No. VG-10 PROV).

The six (6) above-identified patent applications are hereby incorporated herein by reference.

### FIELD OF THE INVENTION

This invention relates to surgical methods and apparatus in general, and more particularly to surgical methods and apparatus for fusing sacroiliac joints.

### BACKGROUND OF THE INVENTION

Lower back pain is a common ailment among the population and results in pain and suffering as well as loss of work time. Effective treatments for lower back pain will alleviate considerable patient suffering and provide economic benefits by reducing employee absenteeism. Until recently, many complaints of lower back pain and leg pain have been attributed to herniated discs or other injuries to the spinal column. However, extensive therapy and treatment has often been unsuccessful in alleviating such pain. Recently, it has been found that some of this lower back and leg pain can be attributed to symptomatic sacroiliac joint dysfunction or instability.

The sacroiliac joint is located at the juncture of the ilium, the upper bone of the pelvis, commonly called the hip bone, and the sacrum at the base of the lumbar spine, where it connects with the L5 vertebra. The function of the sacroiliac joint is the transmission of forces from the spine to the lower extremities and vice-versa. The joint is supported by a range of ligaments, including the sacroiliac ligament at the base of the joint and the anterior sacroiliac ligament at the top of the joint.

The sacroiliac joint has a limited range of motion. Nutation, the relative movement between the sacrum and ilium, is typically one to two degrees. Despite the limited range of motion, a patient's sacroiliac joint can become damaged resulting in hypermobility of the joint. Hypermobility is very difficult to diagnose due to the small range of motion. Therefore, lower back pain or leg pain caused by sacroiliac joint dysfunction, e.g. degenerative sacroiliitis, inflammatory sacroiliitis, iatrogenic instability of the sacroiliac joint, osteitis condensans ilii, or traumatic fracture dislocation of the pelvis, often goes misdiagnosed or undiagnosed.

In patients where sacroiliac joint pain is unresponsive to non-operative treatments, e.g. medication, physical therapy, chiropractic care and steroid injections, surgical stabilization is prescribed. Fusion is a surgical treatment to relieve pain generated from joint dysfunction.

Accordingly, it is a general objective of this invention to provide a method to deliver a device for correcting symptomatic sacroiliac joint dysfunction or instability, for enhancing stability for purposes of immobilizing a joint, and for fusing two opposed bone structures across the joint.

### SUMMARY OF THE INVENTION

The long-standing but heretofore unfulfilled need for improved devices and methods for effecting sacroiliac joint fusion is now met by a new, useful, and nonobvious invention.

The present invention includes a surgical kit for use in a method for fusing a sacroiliac joint, preferably including a stabilization implant, a guide pin, a joint locator, dilation tubes, cutting tools such as a reamer or cannulated reamer, a drill bit, a cutter, and a punch, a novel directional cannula, a novel taping cap, a novel drill guide, and a novel implant positioner.

The invention further includes a method for fusing a sacroiliac joint with an implant, preferably comprising the steps of locating the sacroiliac joint, inserting a guide pin or a joint locator into the sacroiliac joint normal to the immediate bone surfaces on either side of the joint, retracting soft tissue via dilation tubes, sliding a cannulated reamer over the guide pin or the joint locator until a distal end of the reamer engages the sacroiliac joint creating a relatively flat graft site, removing the reamer and guide pin or joint locator, inserting a directional cannula into the sacroiliac joint aligning the teeth located on the distal end of the cannula with the plane of the joint, tapping a proximal end of the directional cannula to reversibly secure the alignment teeth into the sacroiliac joint, inserting the drill guide into the directional cannula, inserting a drill bit through the drill guide and drilling a cavity within the sacroiliac joint to a predetermined depth, removing the drill bit from within the drill guide, removing the drill guide from within the directional cannula, inserting the implant through the directional cannula until the distal end of the implant engages the cavity, inserting an implant positioner to seat the implant at a prescribed depth completely within the cavity, proportionately distributed in the sacrum and ilium, removing the implant positioner from within the directional cannula, removing the directional cannula, and removing the dilation tube.

These and other features of the invention will become apparent from the following detailed description of the preferred embodiments of the invention.

The present invention includes a novel apparatus for effecting sacroiliac joint fusion. The novel structure includes a sacroiliac joint stabilization implant for disposition between the opposing articular surfaces of a sacroiliac joint to immobilize the sacroiliac joint and facilitate fusion between the sacrum and ilium.

More particularly, in one form of the present invention, the novel sacroiliac joint stabilization implant includes an elongated body having a distal end, a proximal end and a longitudinal axis extending between the distal end and the proximal end. The elongated body has a cross-sectional profile characterized by a primary axis and a secondary axis, and at least one stabilizer extending radially outwardly from the elongated body in the secondary axis.

The elongated body has a length along the primary axis which is less than the combined width of the sacrum and ilium making up a sacroiliac joint, and at least one stabilizer has a width that is sized to make a press fit into the gap between the sacrum and ilium making up a sacroiliac joint.

A novel method for fusing a sacroiliac joint includes the steps of providing a sacroiliac joint stabilization implant having an elongated body having a distal end, a proximal end and a longitudinal axis extending between the distal end and the proximal end. The method further includes the steps of providing the elongated body with a cross-sectional profile characterized by a primary axis and a secondary axis and providing at least one stabilizer that extends radially outwardly from the elongated body in the secondary axis.

The method steps further include the steps of forming the elongated body so that it has a length along the primary axis which is less than the combined width of the sacrum and the ilium making up a sacroiliac joint and forming the at least one stabilizer so that it has a width sized to make a press fit into the gap between the sacrum and ilium making up a sacroiliac joint.

Further method steps include the steps of deploying the sacroiliac joint stabilization implant in the sacroiliac joint so that the elongated body is simultaneously positioned within the sacrum and ilium of the sacroiliac joint and so that the at least one stabilizer is positioned within the gap between the sacrum and ilium and maintaining the sacroiliac joint stabilization implant in such position while fusion occurs.

Still further steps include deploying the stabilization implant in the joint so that the elongated body is simultaneously positioned within both of the bones of the joint and at least one stabilizer is positioned within the gap between the bones and maintaining the stabilization implant in this position while fusion occurs.

In a preferred form of the invention, there is provided a stabilization implant comprising:
an elongated body having a distal end, a proximal end and a longitudinal axis extending between the distal end and the proximal end, the elongated body being characterized by a superior body surface and an inferior body surface;
a superior stabilizer extending outwardly from the superior body surface, the superior stabilizer being characterized by a first lateral superior stabilizer surface and a second lateral superior stabilizer surface; and
an inferior stabilizer extending outwardly from the inferior body surface, the inferior stabilizer being characterized by a first lateral inferior stabilizer surface and a second lateral inferior stabilizer surface;
wherein at least one of the first lateral superior stabilizer surface, the second lateral superior stabilizer surface, the first lateral inferior stabilizer surface, and the second lateral inferior stabilizer surface comprise at least one outwardly-extending tab.

In another preferred form of the invention, there is provided a method of positioning an implant in a sacroiliac joint, the method comprising:
providing a stabilization implant comprising:
   an elongated body having a distal end, a proximal end and a longitudinal axis extending between the distal end and the proximal end, the elongated body being characterized by a superior body surface and an inferior body surface;
   a superior stabilizer extending outwardly from the superior body surface, the superior stabilizer being characterized by a first lateral superior stabilizer surface and a second lateral superior stabilizer surface; and
   an inferior stabilizer extending outwardly from the inferior body surface, the inferior stabilizer being characterized by a first lateral inferior stabilizer surface and a second lateral inferior stabilizer surface;
   wherein at least one of the first lateral superior stabilizer surface, the second lateral superior stabilizer surface, the first lateral inferior stabilizer surface, and the second lateral inferior stabilizer surface comprise at least one outwardly-extending tab;
forming an implant cavity for receiving the elongated body of the stabilization implant, wherein forming the implant cavity comprises forming a first cavity in an ilium of the sacroiliac joint and forming a second cavity in a sacrum of the sacroiliac joint, with the implant cavity spanning the sacroiliac joint; and
inserting the elongated body of the stabilization implant into the implant cavity.

In another preferred form of the invention, there is provided a stabilization implant comprising:
an elongated body having a distal end, a proximal end and a central lumen extending between the distal end and the proximal end;
wherein the distal end of the elongated body terminates in a circumferentially-extending distal end cutting surface.

In another preferred form of the invention, there is provided a method of positioning an implant in a sacroiliac joint, the method comprising:
providing a stabilization implant comprising:
   an elongated body having a distal end, a proximal end and a central lumen extending between the distal end and the proximal end;
   wherein the distal end of the elongated body terminates in a circumferentially-extending distal end cutting surface; and
inserting the elongated body of the stabilization implant into the sacroiliac joint, with a first portion of the stabilization implant disposed in an ilium of the sacroiliac joint and a second portion of the stabilization implant disposed in a sacrum of the sacroiliac joint.

### BRIEF DESCRIPTION OF THE DRAWINGS

These and other objects and features of the present invention will be more fully disclosed by the following detailed description of the preferred embodiments of the invention, which is to be considered together with the accompanying drawings wherein like numbers refer to like parts, and further wherein:

FIG. 1 illustrates a stabilization implant formed in accordance with the present invention;

FIG. 2 is a perspective view of a sacroiliac joint;

FIG. 3A is a close-up perspective view of said sacroiliac joint and a drilled, bored, punched, or cut cavity;

FIG. 3B is a close-up perspective view of said sacroiliac joint and said stabilization implant in the final position in the sacroiliac joint;

FIG. 4 is a perspective view of a guide pin;

FIG. 5 is a perspective view of a joint locator,

FIG. 6 is a perspective view of four dilation tubes of increasing diameters;

FIG. 7 is a perspective view of a cannulated reamer;

FIG. 8 is a perspective view of a novel directional cannula;

FIG. 9 is a perspective view of a novel tapping cap:

FIG. 10 is a perspective view of a novel drill guide:

FIG. 11 is a perspective view of a drill bit;

FIG. 12 is a perspective view of a novel implant positioner,

FIG. 13 is a perspective view of said dilation tubes positioned over said sacroiliac joint;

FIG. 14 is a perspective view of a said dilation tubes with said joint locator ensleeved within a lumen of the smallest diameter dilation tube;

FIG. 15 is a perspective view of said cannulated reamer ensleeved within the lumen of the largest diameter dilation tube, sliding over said joint locator,

FIG. 16 is a perspective view of a relatively flat graft site created horizontal to said sacroiliac joint;

FIG. 16A is a longitudinal sectional view of said graft site of FIG. 16;

FIG. 17 is a perspective view of said directional cannula ensleeved within the lumen of said largest diameter dilation tube;

FIG. 17A is a longitudinal sectional view of the distal end of the said directional cannula positioned in the sacroiliac joint of FIG. 17;

FIG. 18 is a perspective view of said drill guide ensleeved in the lumen of said directional cannula with a non-centered guide hole positioned over an ilium bone;

FIG. 19 is a perspective view of said drill guide rotated 180 degrees and subsequently ensleeved in the lumen of said directional cannula with said non-centered guide hole now positioned over a sacrum bone:

FIG. 20 is a perspective view of said sacrum and ilium bones and a drilled, bored, punched, or cut cavity formed in said sacroiliac joint;

FIG. 21 is an exploded perspective view of said stabilization implant being loaded into said directional cannula;

FIG. 22 is a perspective view of said implant and said implant positioner ensleeved in the lumen of the directional cannula prior to final position;

FIG. 23 is a perspective view of said stabilization implant in the final position in said sacroiliac joint;

FIGS. 24 and 25 are schematic perspective views of an alternative novel stabilization implant formed in accordance with the present invention;

FIG. 26 is a schematic side view of the novel stabilization implant of FIGS. 24 and 25;

FIG. 27 is a schematic front view of the novel stabilization implant of FIGS. 24 and 25;

FIG. 28 is a schematic rear view of the novel stabilization implant of FIGS. 24 and 25;

FIG. 29 is a schematic top view of the novel stabilization implant of FIGS. 24 and 25;

FIG. 30 is a schematic perspective view of an alternative novel stabilization implant formed in accordance with the present invention; and

FIG. 31 is a schematic front view of the novel stabilization implant of FIG. 30.

### DETAILED DESCRIPTION OF THE PREFERRED EMBODIMENTS

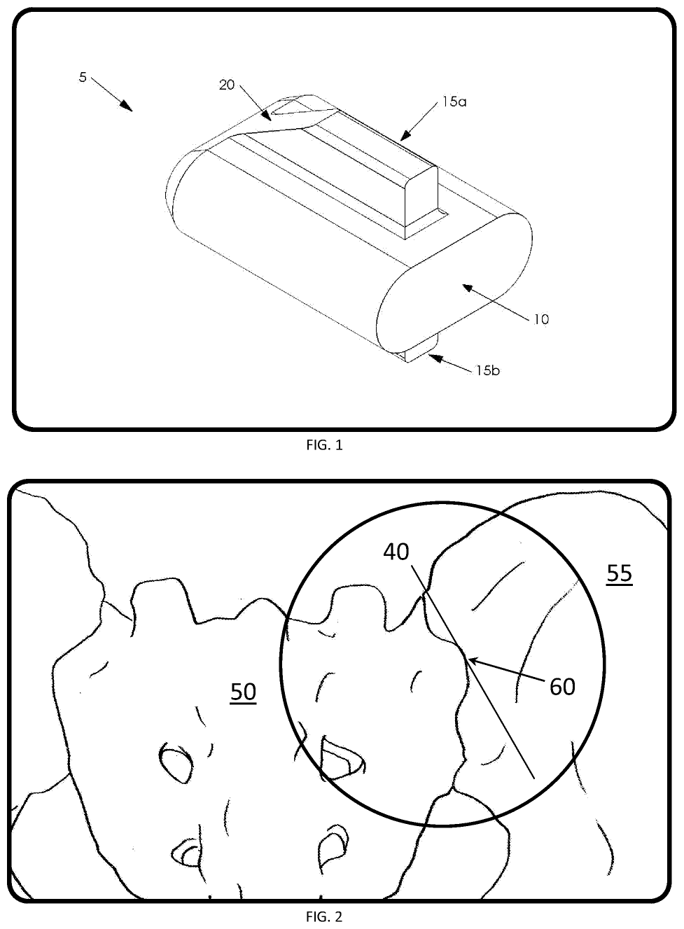

Referring now to FIG. 1, it will be seen that the novel sacroiliac stabilization implant, disclosed further in U.S. Pat. No. 8,162,981 to Vestgaarden, entitled “Method and Apparatus for Spinal Facet Fusion,” incorporated herein by reference, is denoted as a whole by the reference numeral 5. Stabilization implant 5 generally includes body 10 and at least one stabilizer 15.

Body 10 is an elongated element having structural integrity. Preferably the distal end of body 10 (and the distal end of stabilizer 15 as well) is chamfered as shown at 20 to facilitate insertion of fusion implant 5 into the sacroiliac joint. Preferably, as depicted in FIG. 1, body 10 has a rounded rectangular cross-section, or an ovoid cross-section, a laterally-extended cross-section, or some other non-round cross-section, so as to inhibit rotation of body 10 about a longitudinal center axis.

At least one stabilizer 15 is preferably received in the gap located between the opposing sacroiliac surfaces to prevent rotation of stabilization implant 5 within the sacroiliac joint. In one preferred embodiment of the invention, two stabilizers 15a and 15b are provided, one disposed along the upper surface of body 10 and one disposed along the lower surface of body 10. Stabilizers 15 preferably have a width just slightly larger than the gap between the opposing articular surfaces of a sacroiliac joint so that the stabilizers can make a snug fit therebetween.

Stabilization implant 5 is inserted into a sacroiliac joint using a posterior approach. The posterior approach is familiar to spine surgeons, thereby providing an increased level of comfort for the surgeon.

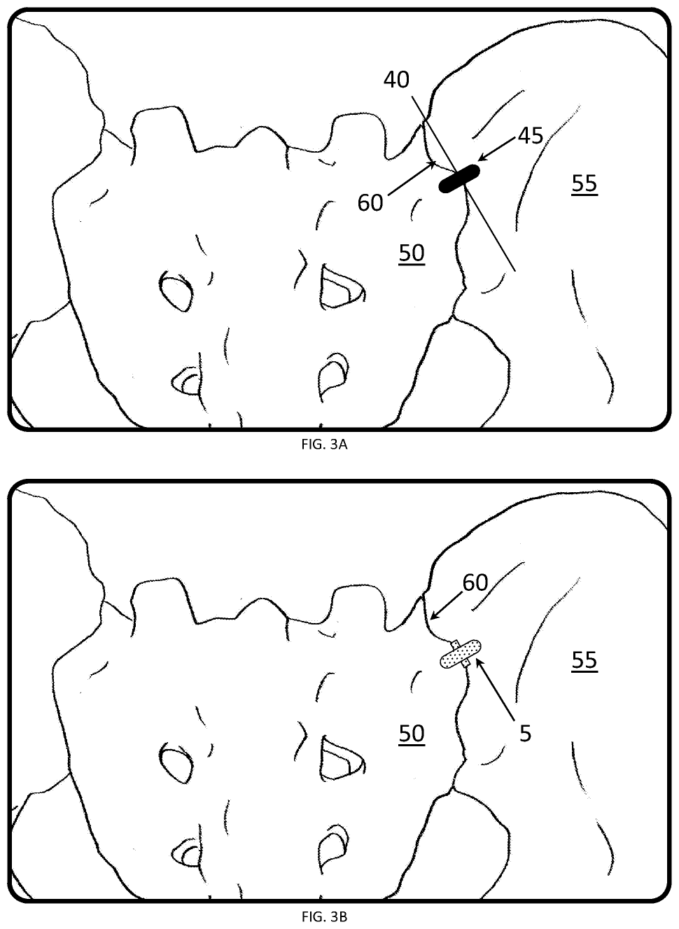

In use, and referring now at FIG. 2, an instrument is first used to determine plane 40 of sacroiliac joint 60. Identifying the plane of the sacroiliac joint is important, since this is used to identify the proper position for cavity 45 (FIG. 3A) which is to be formed across the sacroiliac joint to receive stabilization implant 5.

At least one of the instruments includes a directional feature which is used to maintain the alignment of the instrumentation with the plane of the sacroiliac joint. A directional cannula may include a flat portion and the remaining instruments may include a flat portion on an opposite portion of the instrument so that the instruments may only be inserted through the cannula at zero degrees (0°), one hundred eighty degrees (180°), or both.

The directional cannula provides the passageway for the placement and insertion of a stabilization device, as well as for performing drilling/cutting or other preparatory work for appropriate stabilization device embodiments.

The directional cannula can have an interior central passage of a circular cross section, oval cross section, rectangular cross section or other desired shape that provides the desired guide channel to deliver a stabilization device into cavity 45.

After the proper position for cavity 45 has been identified, a drill (or reamer, punch, dremel, router, burr, etc.) is used to form cavity 45 in sacroiliac joint 60. Cavity 45 is formed across plane 40 so that substantially one-half of cavity 45 is formed in sacrum 50, and substantially one-half is formed in ilium 55.

After cavity 45 has been formed in (or, perhaps more literally, across) the sacroiliac joint 60, and now referring to FIG. 3B, stabilization implant 5 is inserted into cavity 45. More particularly, stabilization implant 5 is inserted into cavity 45 so that (i) main body 10 spans the gap between opposing sacrum 50 and ilium 55, and (ii) stabilizers 15 extend between the opposing sacrum and ilium surfaces. Preferably, stabilization implant 5 is slightly oversized relative to cavity 45 so as to create a press fit. Stabilization implant 5 provides the stability and strength needed to immobilize the sacroiliac joint 60 while fusion occurs. Due to the positioning of stabilizers 15 between the opposing sacrum and ilium surfaces, and due to the non-circular cross-section of main body 10, stabilization implant 5 is held against rotation within cavity 45, which will in turn holds sacrum 50 and ilium 55 stable relative to one another.

### Detailed Surgical Technique

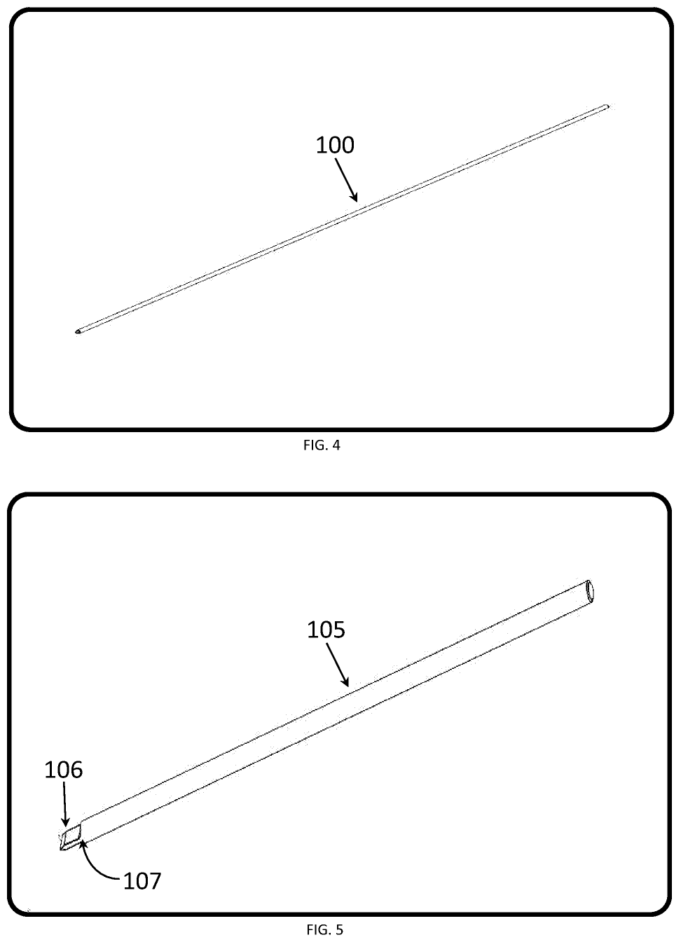

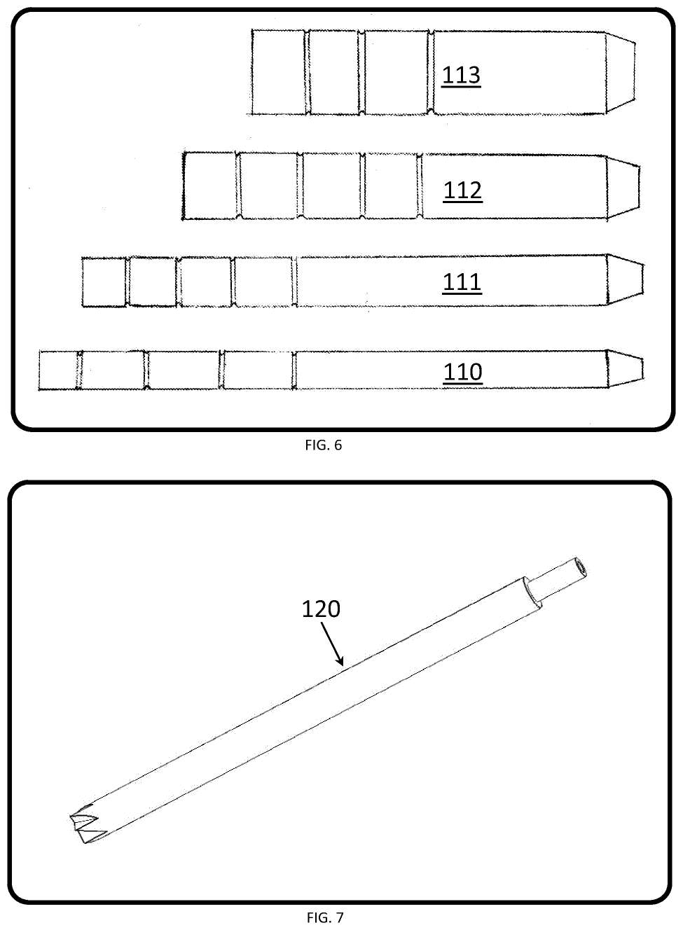

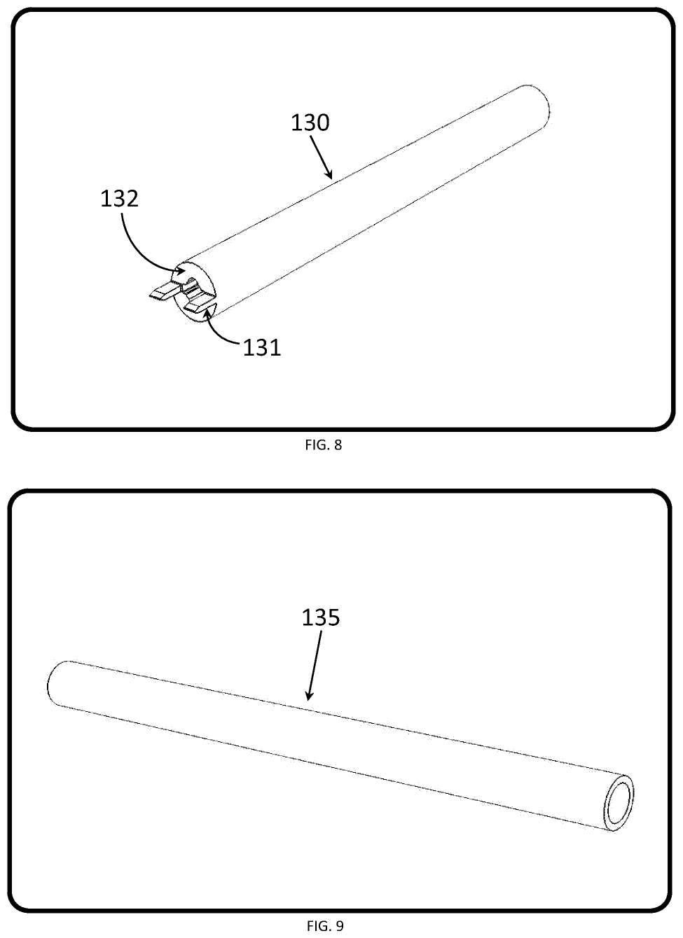

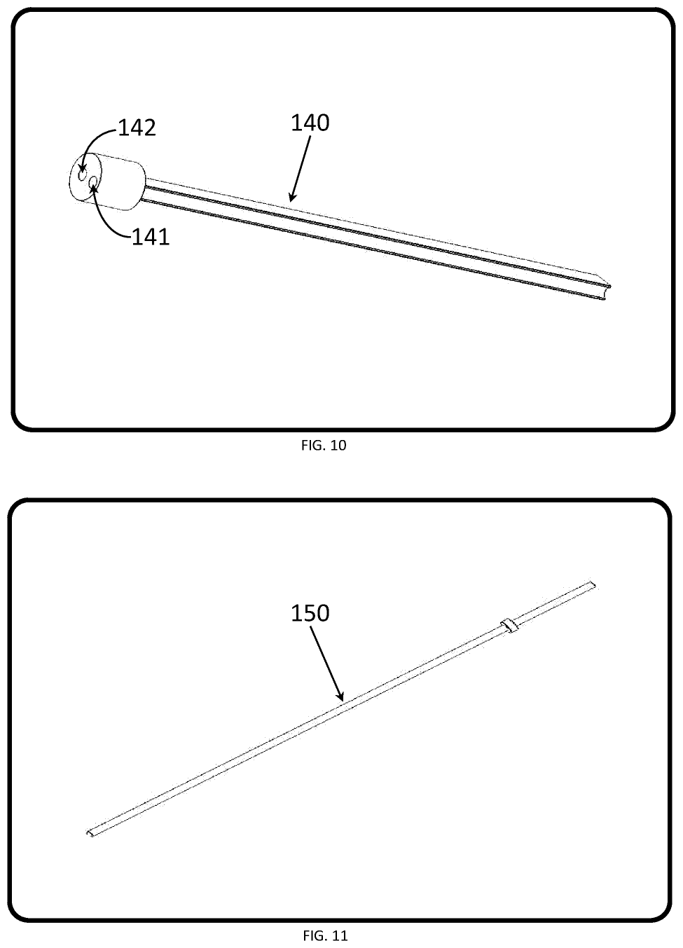

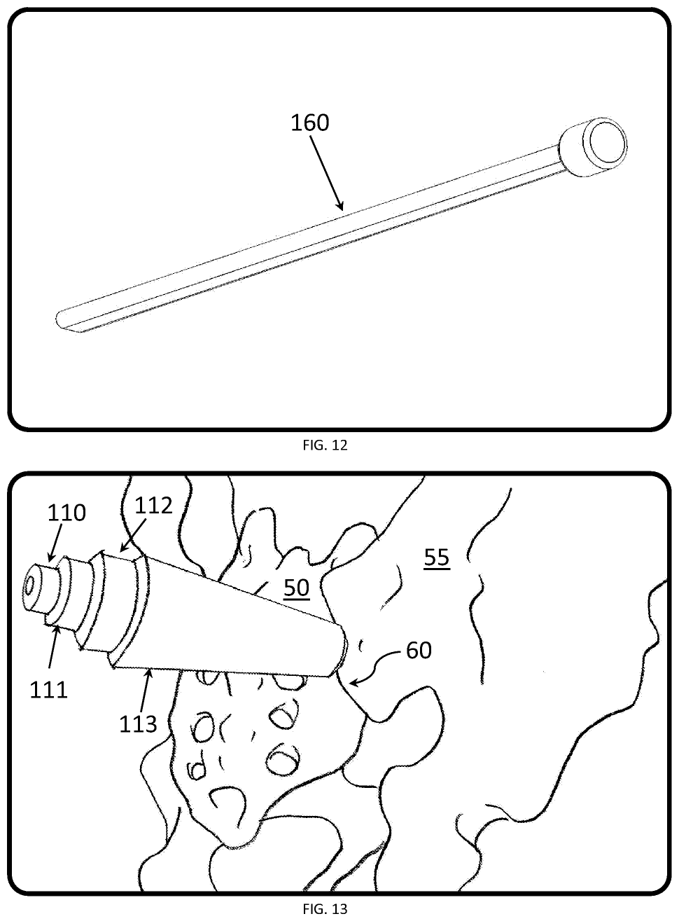

A preferred surgical technique for using stabilization implant 5 employs guide pin 100 (FIG. 4), joint locator 105 (FIG. 5), dilation tubes 110-113 (FIG. 6), cannulated reamer 120 (FIG. 7), directional cannula 130 (FIG. 8), tapping cap 135 (FIG. 9), drill guide 140 (FIG. 10), drill bit 150 (FIG. 11), and implant positioner 160 (FIG. 12).

First, the sacroiliac joint is localized indirectly by fluoroscopy, or directly by visualization during an open procedure. A path through soft tissue to the sacroiliac joint is then created via surgeon's preference, such as open, minimally-invasive, percutaneous, or arthroscopic.

A set of dilation tubes 110-113 (FIG. 13) having increasing diameters is then inserted into the soft tissue opening in sequence of increasing diameters to sufficiently retract the soft tissue exposing a graft site.

Next, joint locator 105 (FIG. 14) is slid into a lumen of dilation tube 110 until blade 106 engages sacroiliac joint 60 and is aligned with joint plane 40. Then joint locator 105 is lightly tapped so as to insert joint locator blade 106 into sacroiliac joint 60 until positive stop 107 is engaged.

Next, internal dilation tubes 110-112 are removed from within the lumen of dilation tube 113.

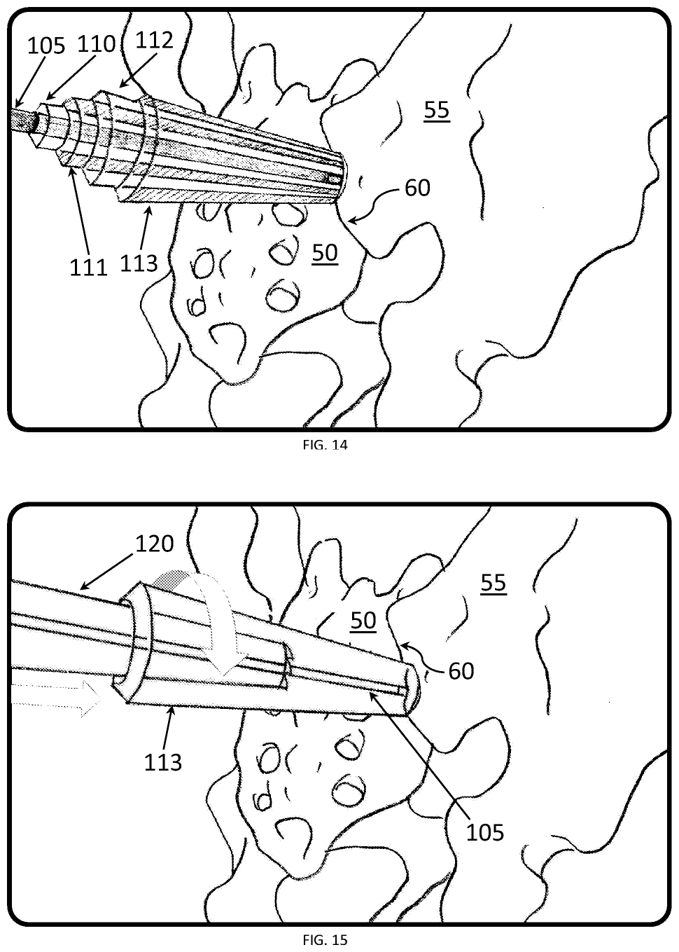

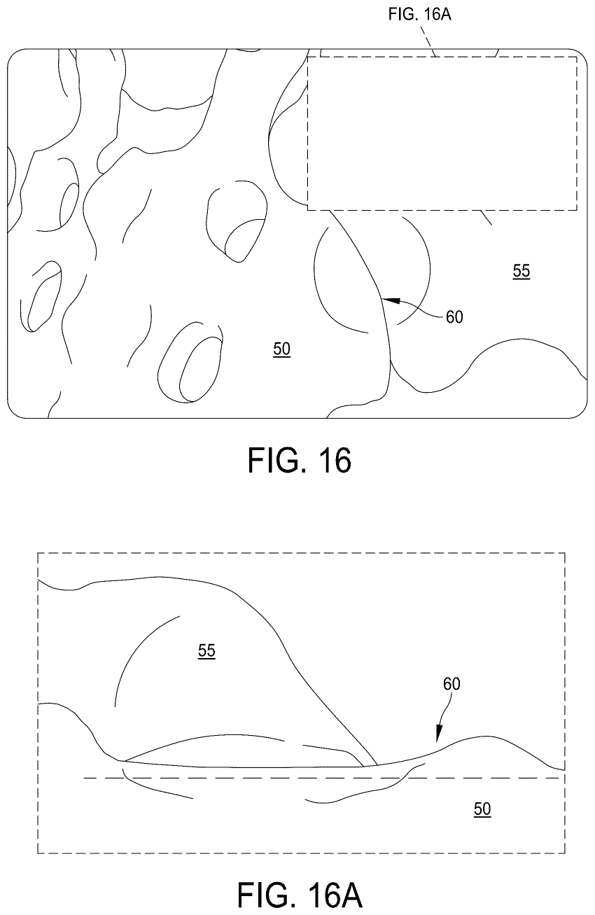

Cannulated reamer 120 is then slid over joint locator 105 to remove any bone obstructing the joint and to prepare the graft surface for receiving directional cannula 130 and stabilization implant 5 (FIG. 15). The distal end of reamer 120 is advanced until it sufficiently engages sacroiliac joint 60, thereby preparing a relatively flat graft surface perpendicular to sacroiliac joint 60 (FIGS. 16 and 16A). The position of reamer 120 and joint locator 105 is verified by viewing the coronal and sagittal planes.

Reamer 120 and joint locator 105 are then removed from within the lumen of dilation tube 113.

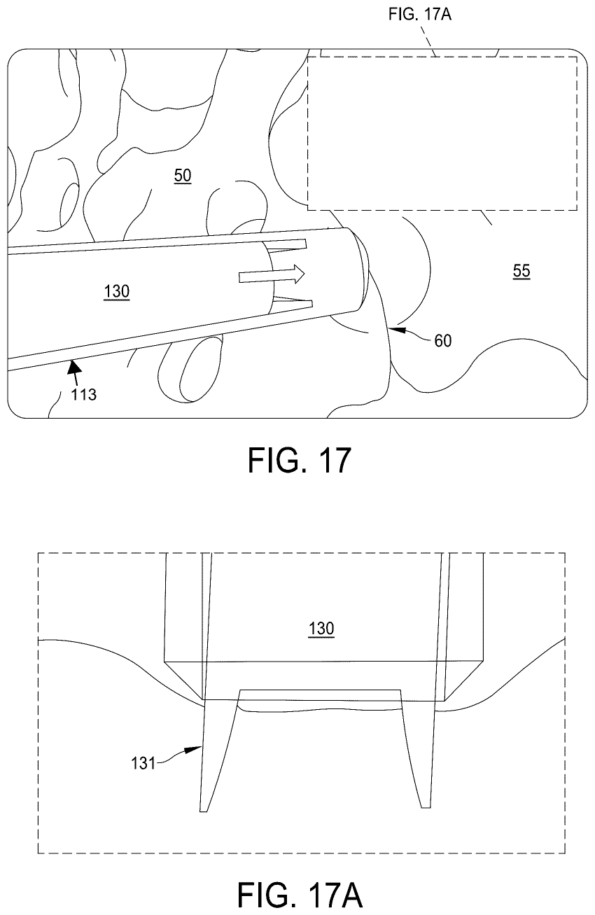

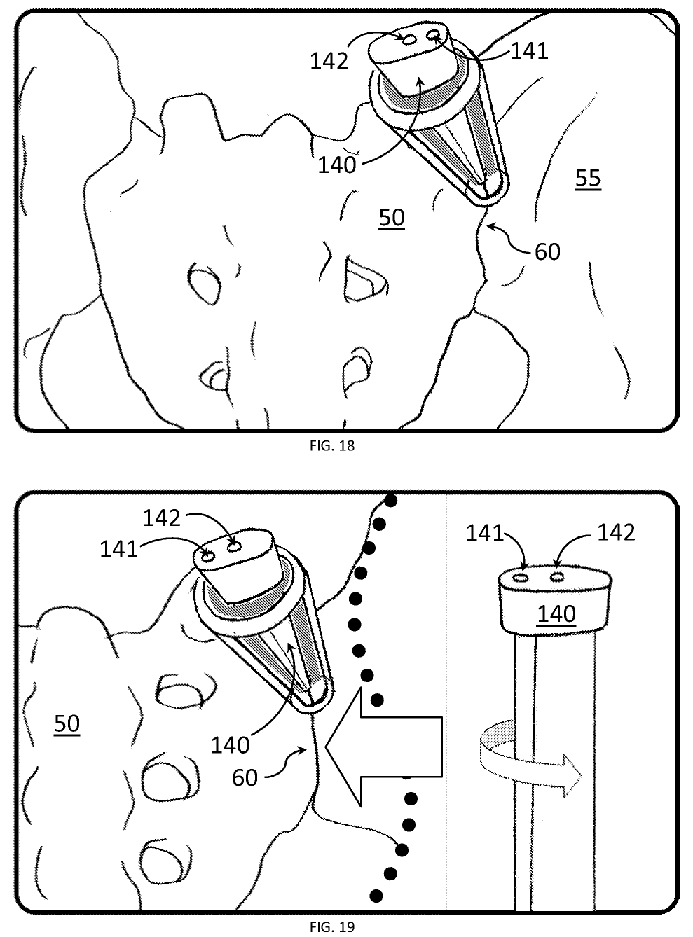

Next, directional cannula 130 is inserted into the lumen of dilation tube 113 until a distal end of cannula 130 engages sacroiliac joint 60 (FIG. 17). Directional cannula teeth 131 are then aligned with plane 40 of sacroiliac joint 60. Once teeth 131 of cannula 130 are aligned with plane 40, directional cannula 130 is lightly tapped to insert cannula teeth 131 into sacroiliac joint 60 until positive stop 132 engages sacroiliac joint 60 (FIG. 17A).

Drill guide 140 is then inserted into a lumen of directional cannula 130 with non-centered guide hole 141 positioned over iliac bone 55 (FIG. 18). Drill guide 140 is advanced within the lumen of directional cannula 130 until drill guide 140 reaches a mechanical stop on directional cannula 130. Then, with drill guide 140 in place, irrigation fluid (e.g., a few drops of saline) is placed into the drill guide hole 141 positioned over iliac bone 55. Next, drill bit 150 is inserted into guide hole 141 and used to drill a cavity in iliac bone 55. Drilling continues until drill bit 150 reaches a mechanical stop on drill guide 140. Then drill bit 150 is removed from the lumen of guide hole 141. Next, with drill guide 140 remaining in position, irrigation fluid (e.g., a few drops of saline) is placed into central guide hole 142 of drill guide 140. Drill bit 150 is then inserted in a lumen of guide hole 142 and used to drill a cavity in sacroiliac joint 60, between sacrum 50 and ilium 55. Next, drill bit 150 is removed from the lumen of guide hole 142. Drill guide 140 is then removed from the lumen of directional cannula 130.

Drill guide 140 is rotated 180 degrees, and is reinserted into the lumen of directional cannula 130 in order to drill sacrum 50 (FIG. 19). With drill guide 140 in place, irrigation fluid (e.g., a few drops of saline) is placed into drill guide hole 141, now positioned over sacrum 50. Next, drill bit 150 is inserted into the lumen of guide hole 141 and used to drill a cavity in sacrum bone 50. Drilling continues until drill bit 150 reaches a mechanical stop on drill guide 140. Then drill bit 150 is removed from the lumen of guide hole 141. Next, with drill guide 140 remaining in position, irrigation fluid (e.g., a few drops of saline) is placed into central guide hole 142 of drill guide 140. Next, drill bit 150 is inserted into guide hole 142 and used to drill a cavity in sacroiliac joint 60, between sacrum 50 and ilium 55. Next, drill bit 150 is removed from guide hole 142 and drill guide 140 is removed from the lumen of directional cannula 130.

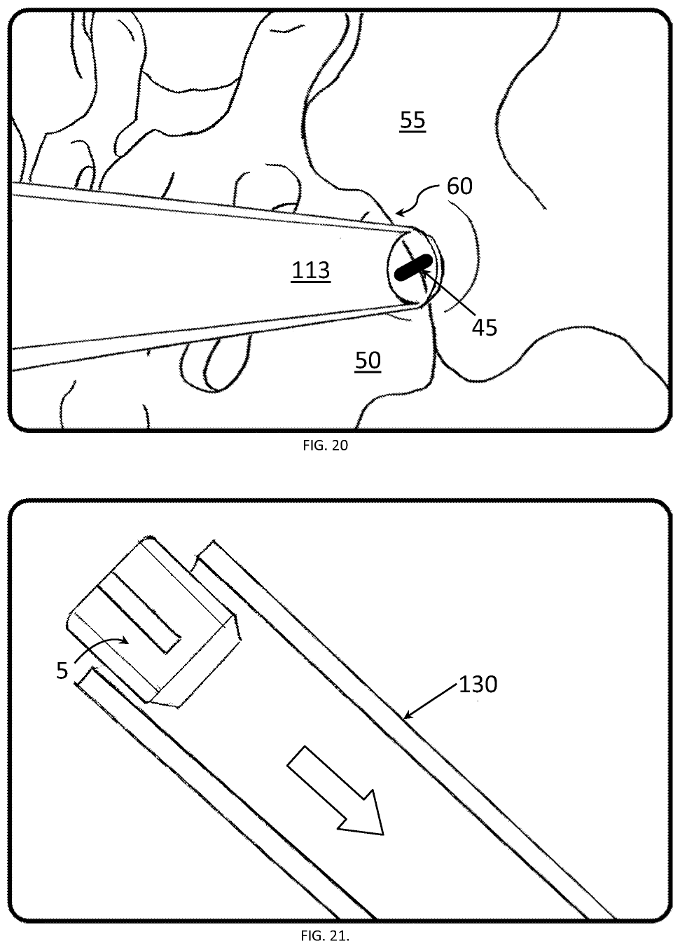

This procedure creates cavity 45 (FIG. 20) that is sufficiently deep and that is proportionately distributed in sacrum 50 and ilium 55 to receive stabilization implant 5.

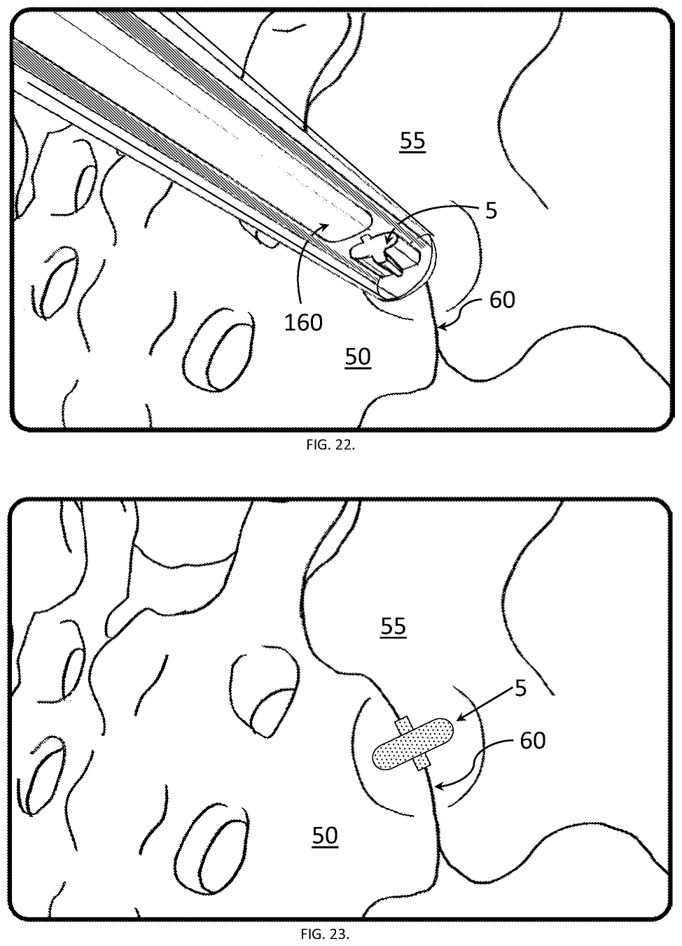

Stabilization implant 5 is then inserted, distal end first, into the lumen of directional cannula 130 (FIG. 21). Next, implant positioner 160 is inserted into the lumen of directional cannula 130 and advanced until resistance is felt, indicating that the distal end of implant 5 has engaged cavity 45 (FIG. 22). Next, implant positioner 160 is lightly tapped to drive implant 5 into cavity 45 created laterally across sacroiliac joint 60 (FIG. 23). Stabilization implant 5 is preferably countersunk 1-2 mm into sacroiliac joint 60.

Finally, implant positioner 160 and directional cannula 130 are removed from the lumen of dilation tube 113. Dilation tube 113 is then removed from the soft tissue and the incision is closed.

It will be appreciated that, if desired, multiple stabilization implants 5 (e.g., three stabilization implants 5) may be inserted into each of the sacroiliac joints 60 (i.e., the current sacroiliac joint 60 and the contralateral sacroiliac joint 60) in order to achieve the desired degree of joint immobilization. Where multiple stabilization implants 5 are to be used to stabilize sacroiliac joint(s) 60, the foregoing steps are repeated for additional locations in the current sacroiliac joint 60 and in contralateral sacroiliac joint 60.

### Alternative Surgical Technique

First, sacroiliac joint 60 is localized indirectly by fluoroscopy, or directly by visualization during an open procedure. Guide pin 100 is inserted into sacroiliac joint 60, normal to immediate opposing joint surfaces when sacroiliac joint 60 is exposed, or drilled into joint 60 through the iliac crest when sacroiliac joint 60 is obstructed. The position of guide pin 100 is determined by viewing the coronal and sagittal planes. Guide pin 100 is then lightly tapped to insert guide pin 100 approximately 15-20 mm into sacroiliac joint 60, along joint plane 40 (FIG. 3A).

Next, a set of dilation tubes 110-113 (FIG. 6) having increasing diameters is slid over guide pin 100 into the soft tissue in sequence of increasing diameters to sufficiently retract soft tissue exposing a graft site (FIG. 13). Once a sufficient surgical area is exposed, internal dilation tubes 110-112 are removed from within the lumen of dilation tube 113. Next, referring to FIG. 15, cannulated reamer 120 is slid over guide pin 100 within the lumen of dilation tube 113 to remove any bone obstructing sacroiliac joint 60 and to prepare the graft surface for receiving directional cannula 130 and fusion implant 5. The distal end of reamer 120 is advanced until it sufficiently engages sacroiliac joint 60, thereby preparing a relatively flat graft surface perpendicular to sacroiliac joint 60 (FIGS. 16A and 16B). The position of reamer 120 is verified by viewing the coronal and sagittal planes.

Next, reamer 120 and guide pin 100 are removed from within the lumen of dilation tube 113.

The steps disclosed above in the section entitled “Detailed Surgical Technique” are then followed to complete the procedure.

Numerous advantages are achieved by the present invention. For example, the present invention provides a fast, simple, minimally-invasive and easily reproduced approach for effecting sacroiliac joint fusion.

While stabilization implant 5 has been disclosed above in the context of fusing a sacroiliac joint, it should also be appreciated that stabilization implant 5 may be used to stabilize and fuse any joint having anatomy similar to the sacroiliac joint, i.e., a pair of opposing bony surfaces defining a gap therebetween, with the stabilizer of the stabilization implant being sized to be positioned within the gap. By way of example but not limitation, stabilization implant 5 may be used in small joints such as the fingers, toes, etc.

### Alternative Stabilization Implant

In the foregoing description there is described a novel stabilization implant 5 configured for insertion into a sacroiliac joint 60 in the joint space between a sacrum 50 and an ilium 55, whereby to stabilize the SI joint so as to promote bone fusion across the joint space.

In another embodiment of the present invention, if desired, an alternative novel stabilization implant comprising elements to enhance stability of sacrum 50 relative to ilium 55 when implanted into sacroiliac joint 60 may be used in place of, or in addition to, the aforementioned stabilization implant 5, whereby to promote bone fusion across the joint space.

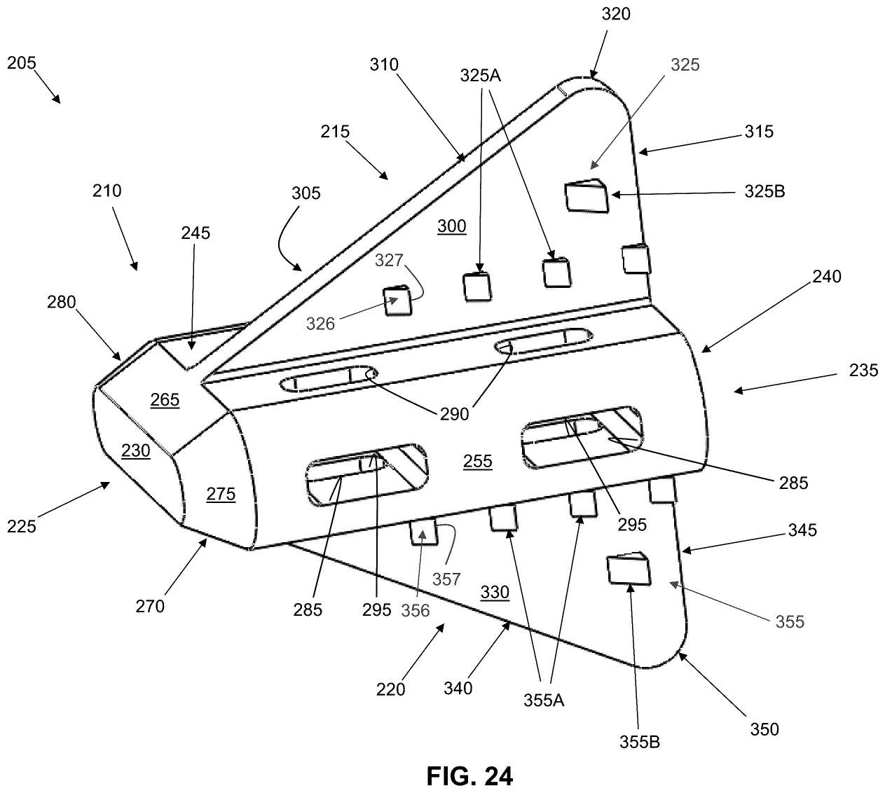

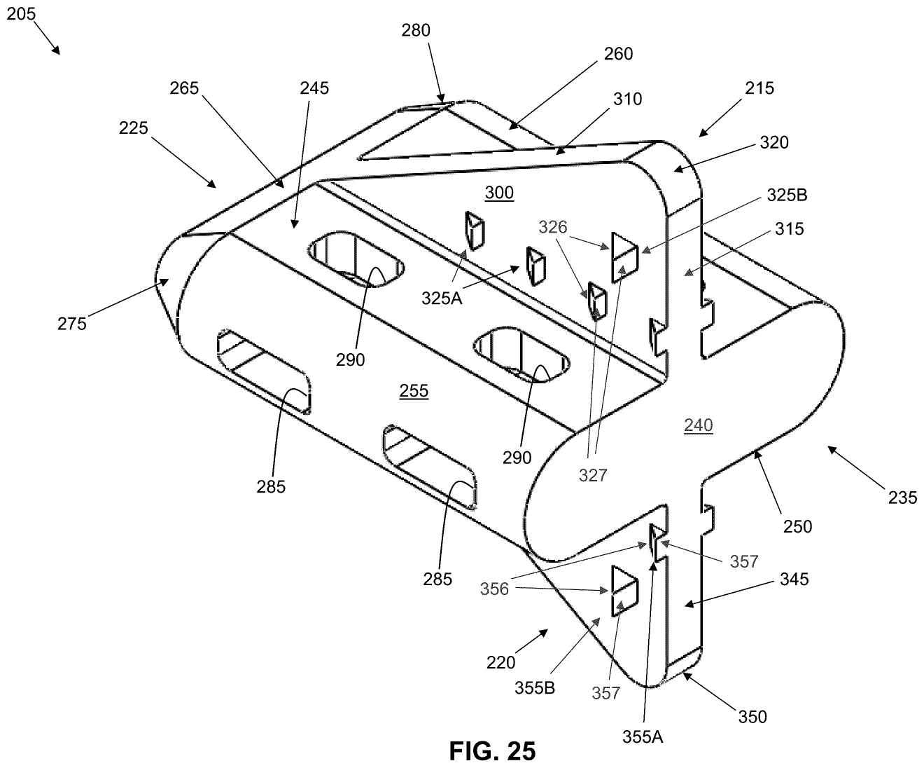

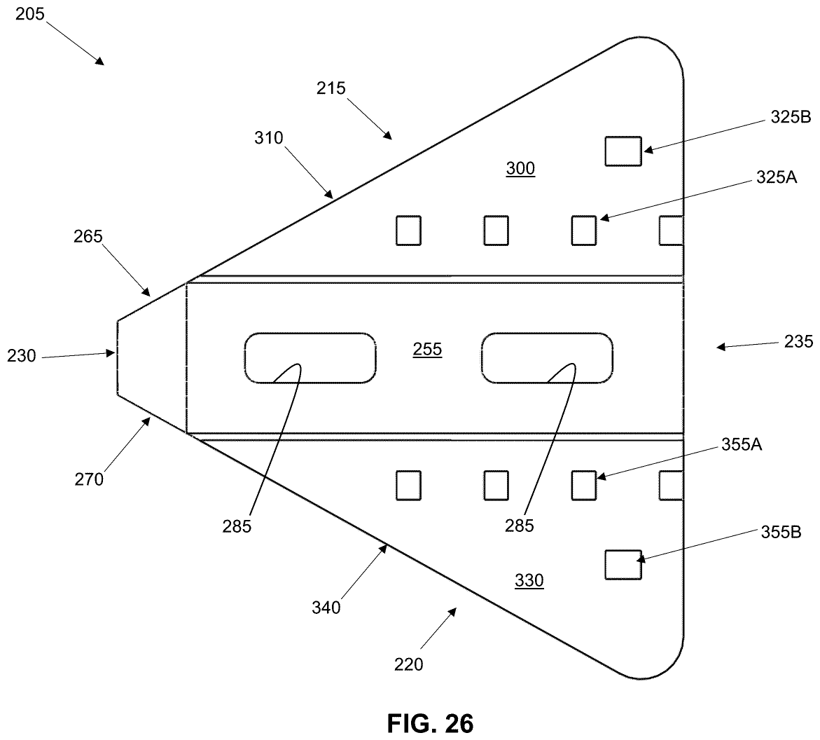

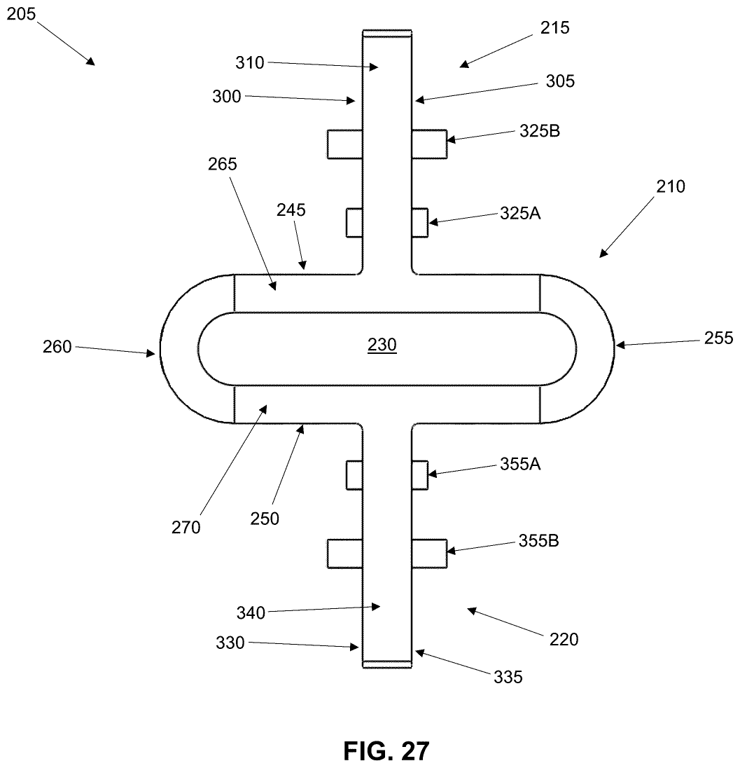

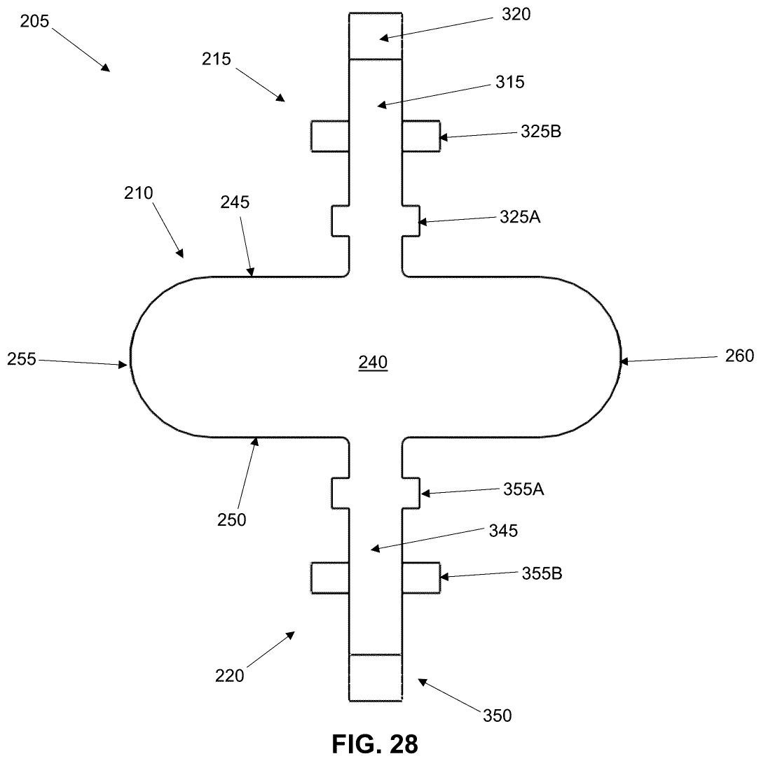

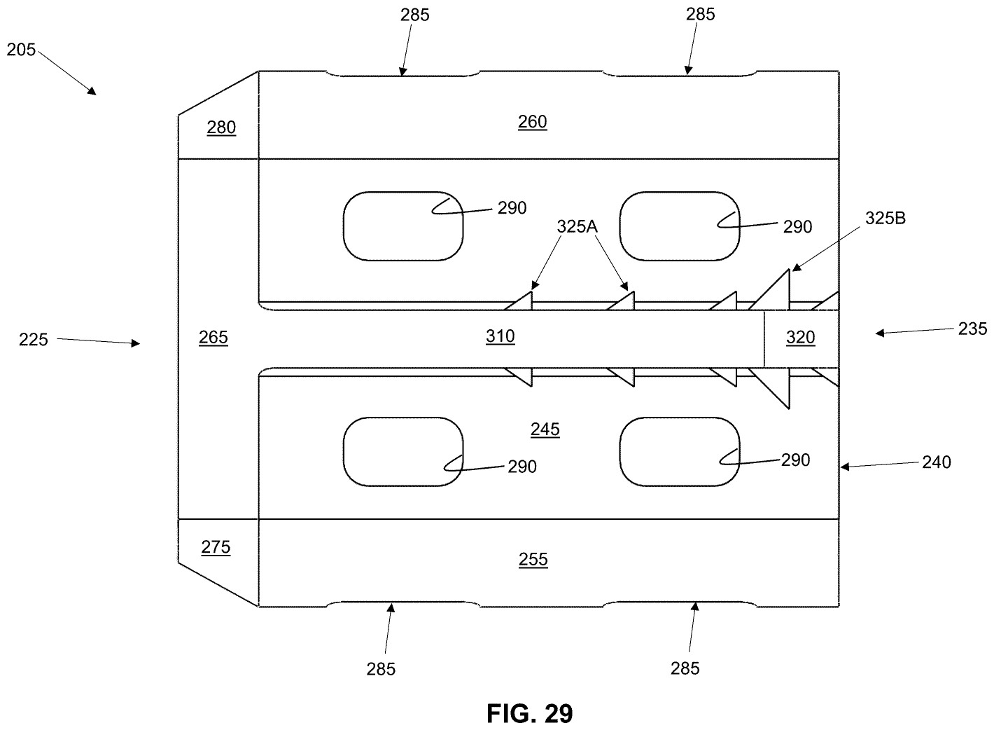

More particularly, and looking now at FIGS. 24-29, there is shown an alternative novel stabilization implant 205. Stabilization implant 205 generally comprises a body 210, a superior stabilizer 215 and an inferior stabilizer 220.

Body 210 generally comprises a rounded rectangular cross-section, an ovoid cross-section, a laterally-extended cross-section, or such other non-round cross-section as will inhibit rotation of body 210 about its central longitudinal axis when stabilization implant 205 is disposed in sacroiliac joint 60, as will hereinafter be discussed in further detail. More particularly, body 210 comprises a distal end 225 characterized by a planar distal end surface 230, a proximal end 235 characterized by a planar proximal end surface 240, a superior surface 245, an inferior surface 250, a first lateral surface 255 and a second lateral surface 260.

In one preferred form of the invention, a superior chamfer surface 265 extends between superior surface 245 and distal end surface 230, an inferior chamfer surface 270 extends between inferior surface 250 and distal end surface 230, a first lateral chamfer surface 275 extends between first lateral surface 255 and distal end surface 230, and a second lateral chamfer surface 280 extends between second lateral surface 260 and distal end surface 230. As a result of this construction, it will be appreciated that distal end 225 of stabilization implant 205 comprises a tapered distal end for facilitating insertion into cavity 45 formed in sacroiliac joint 60.

In one preferred form of the invention, first lateral surface 255 and second lateral surface 260 comprise curved surfaces, whereby to provide body 210 of stabilization implant 205 with a generally ovoid cross-section that matches the cross-section of the bone cavity in which stabilization implant 205 is to be inserted, as will hereinafter be discussed in further detail below.

If desired, body 210 may comprise one or more lateral windows 285 opening on one or both of first lateral surface 255 and second lateral surface 260 for facilitating bony in-ingrowth into stabilization implant 205 and/or for carrying bone-promoting material. The one or more lateral windows 285 may pass partly into body 210, or may pass entirely through body 210 (such that a lateral window 285 opening on first lateral surface 255 also opens on second lateral surface 260). Alternatively and/or additionally, if desired, body 210 may comprise one or more superior windows 290 opening on superior surface 245, and/or one or more inferior windows 295 opening on inferior surface 250 for facilitating bony in-ingrowth into stabilization implant 205 and/or for carrying bone-promoting material. It should be appreciated that, if desired, one or more superior windows 290 and/or inferior windows 295 may open onto the one or more passageways defined by lateral windows 285.

Superior stabilizer 215 generally comprises a first lateral stabilizer surface 300, a second lateral stabilizer surface 305, and an angled superior stabilizer surface 310. The distance between first lateral stabilizer surface 300 and second lateral stabilizer surface 305 (i.e., the width of superior stabilizer 215) comprises a width slightly smaller than, equal to, or just slightly larger than the gap between the opposing articular surfaces of the joint into which stabilizer implant 205 is to be inserted. By way of example but not limitation, where stabilizer implant 205 is to be disposed in a cavity 45 formed in sacroiliac joint 60, the distance between first lateral stabilizer surface 300 and second lateral stabilizer surface 305 preferably comprises a width just slightly larger than the gap between the opposing articular surfaces of the ilium and the sacrum such that superior stabilizer 215 can make a snug fit (e.g., a “press-fit”) therebetween. Superior stabilizer 215 also comprises a proximal superior stabilizer surface 315 which is preferably disposed co-planar with planar proximal end surface 240 of body 210. A curved superior stabilizer surface 320 extends between angled superior surface 310 and proximal superior stabilizer surface 315. If desired, angled superior stabilizer surface 310 of superior stabilizer 215 may be angled so as to be co-planar with superior chamfer surface 265. Alternatively, if desired, the angle of angled superior stabilizer surface 310 of superior stabilizer 215 may be different than the angle of superior chamfer surface 265.

In one preferred form of the present invention, a plurality of tabs 325 are formed on one or both of first lateral stabilizer surface 300 and second lateral stabilizer surface 305, whereby to grip the surface of articulating bones disposed on either side of superior stabilizer 215 when stabilization implant 205 is disposed in the gap between the opposing articular surfaces of a joint, as will be apparent to one of skill in the art in view of the present disclosure. Tabs 325 extend laterally outboard of one or both of first lateral stabilizer surface 300 and second lateral stabilizer surface 305. Each tab 325 preferably comprises an angled distal surface 326 for facilitating insertion of stabilization implant 205 into the joint space, and a proximal surface 327 disposed generally perpendicular to lateral stabilizer surfaces 300, 305 in order to prevent retrograde (i.e., proximal) movement of stabilization implant 205 out of the joint space after implantation. The lateral outboard distance to which tabs 325 extend may be varied, with some tabs 325A extending laterally outboard a first distance, and some tabs 325B extending laterally outboard a second distance which is greater than the first distance.

Inferior stabilizer 220 generally comprises a first lateral stabilizer surface 330, a second lateral stabilizer surface 335, and an angled inferior stabilizer surface 340. The distance between first lateral stabilizer surface 330 and second lateral stabilizer surface 335 (i.e., the width of inferior stabilizer 220) comprises a width slightly smaller than, equal to, or just slightly larger than the gap between the opposing articular surfaces of the joint into which stabilizer implant 205 is to be inserted. By way of example but not limitation, where stabilizer implant 205 is to be disposed in a cavity 45 formed in sacroiliac joint 60, the distance between first lateral stabilizer surface 330 and second lateral stabilizer surface 335 comprises a width just slightly larger than the gap between the opposing articular surfaces of the ilium and the sacrum such that inferior stabilizer 220 can make a snug fit (e.g., a “press-fit”) therebetween. Inferior stabilizer 220 also comprises a proximal inferior stabilizer surface 345 which is preferably disposed co-planar with planar proximal end surface 240 of body 210. A curved inferior stabilizer surface 350 extends between angled inferior stabilizer surface 340 and proximal inferior stabilizer surface 345. If desired, angled inferior stabilizer surface 340 of inferior stabilizer 220 may be angled so as to be co-planar with inferior chamfer surface 270. Alternatively, if desired, the angle of angled inferior stabilizer surface 340 of inferior stabilizer 220 may be different than the angle of inferior chamfer surface 270.

In one preferred form of the present invention, a plurality of tabs 355 are formed on one or both of first lateral stabilizer surface 330 and second lateral stabilizer surface 335, whereby to grip the surface of articulating bones disposed on either side of inferior stabilizer 220 when stabilization implant 205 is disposed in the gap between the opposing articular surfaces of a joint, as will be apparent to one of skill in the art in view of the present disclosure. Tabs 355 extend laterally outboard of one or both of first lateral stabilizer surface 330 and second lateral stabilizer surface 335. Each tab 355 preferably comprises an angled distal surface 356 for facilitating insertion of stabilization implant 205 into the joint space, and a proximal surface 357 disposed generally perpendicular to lateral stabilizer surfaces 330, 335 in order to prevent retrograde (i.e., proximal) movement of stabilization implant 205 out of the joint space after implantation. The lateral outboard distance to which tabs 355 extend may be varied, with some tabs 355A extending laterally outboard a first distance, and some tabs 355B extending laterally outboard a second distance which is greater than the first distance.

In use, stabilization implant 205 is inserted into a cavity formed in the articulating surfaces of two adjacent bones that define a joint space. By way of example but not limitation, where implant 205 is to be inserted into the SI joint, cavity 45 is formed using one of the surgical techniques discussed above, with cavity 45 being dimensioned so to be sufficiently deep and proportionally distributed in sacrum 50 and ilium 55 to receive stabilization implant 205 therein. Implant 205 is then inserted into cavity 45 (e.g., using the novel technique discussed above) such that superior stabilizer 215 and inferior stabilizer 220 make a press-fit with articulating surfaces of sacrum 50 and ilium 55. It will be appreciated that superior stabilizer 215 and inferior stabilizer 220 engage the surrounding bone so as to inhibit rotation of body 210 within sacroiliac joint 60, and act to stabilize sacrum 50 relative to ilium 55 such that sacroiliac joint 60 is immobilized, whereby to facilitate joint fusion. It will also be appreciated that tabs 325A, 325B on first lateral stabilizer surface 300 and second lateral stabilizer surface 305 of superior stabilizer 215 and tabs 355A, 355B on first lateral stabilizer surface 330 and second lateral stabilizer surface 335 of inferior stabilizer 220 help to prevent stabilization implant 205 from “backing out” of sacroiliac joint 60 (i.e., retrograde movement) after stabilization implant 205 has been positioned in sacroiliac joint 60. It should also be appreciated that tabs 325A, 325B, 355A, 355B engage the articulating surfaces of sacrum 50 and ilium 55 such that the articulating surfaces do not move relative to stabilization implant 205, thereby stabilizing sacroiliac joint 60 and promoting bone fusion across sacroiliac joint 60. And it should further be appreciated that the provision of lateral windows 285, superior windows 290 and/or inferior windows 295 provide clear avenues for bony in-growth into (or through) stabilization implant 205 across sacroiliac joint 60, further enhancing stabilization of sacroiliac joint 60 (e.g., via bone fusion).

Although stabilization implant 205 has been disclosed above in the context of fusing a sacroiliac joint, it should also be appreciated that stabilization implant 205 may be used to stabilize and fuse any joint having anatomy similar to the sacroiliac joint, i.e., a pair of opposing bony surfaces defining a gap therebetween, with superior stabilizer 215 and inferior stabilizer 220 being sized to be positioned within the joint gap. By way of example but not limitation, stabilization implant 205 may also be used in small joints such as the fingers, toes, etc.

### Alternative Stabilization Implant Comprising Distal Cutting Edge

In still another embodiment of the present invention, if desired, an alternative novel stabilization implant comprising a distal cutting edge to facilitate insertion into the joint space of sacroiliac joint 60 (e.g., to cut into the bone of sacrum 50 and/or ilium 55) may be used in place of, or in addition to, the aforementioned stabilization implants 5 and 205, whereby to promote bone fusion across the joint space.

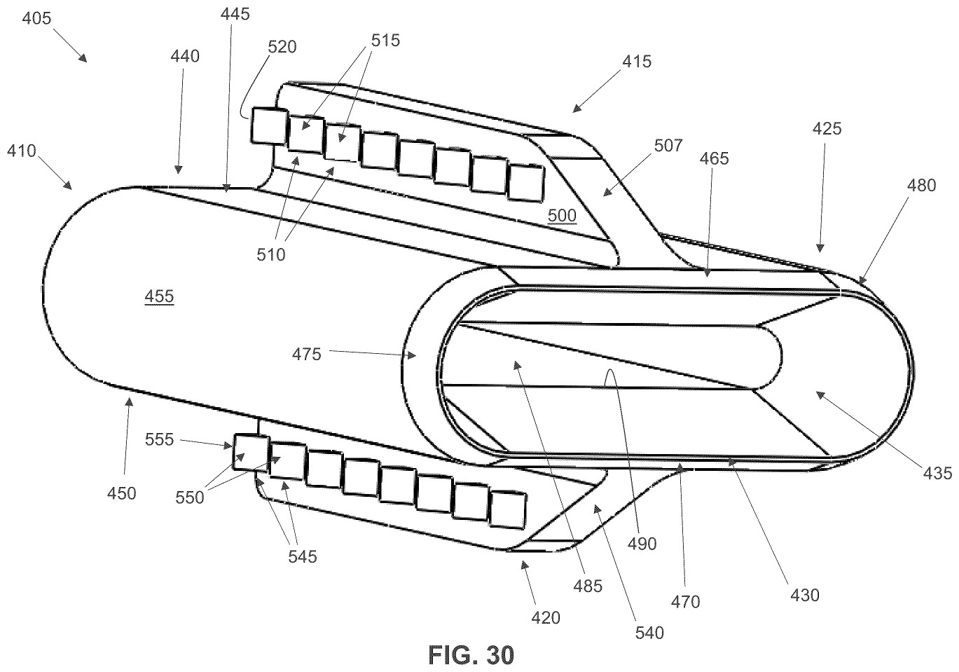

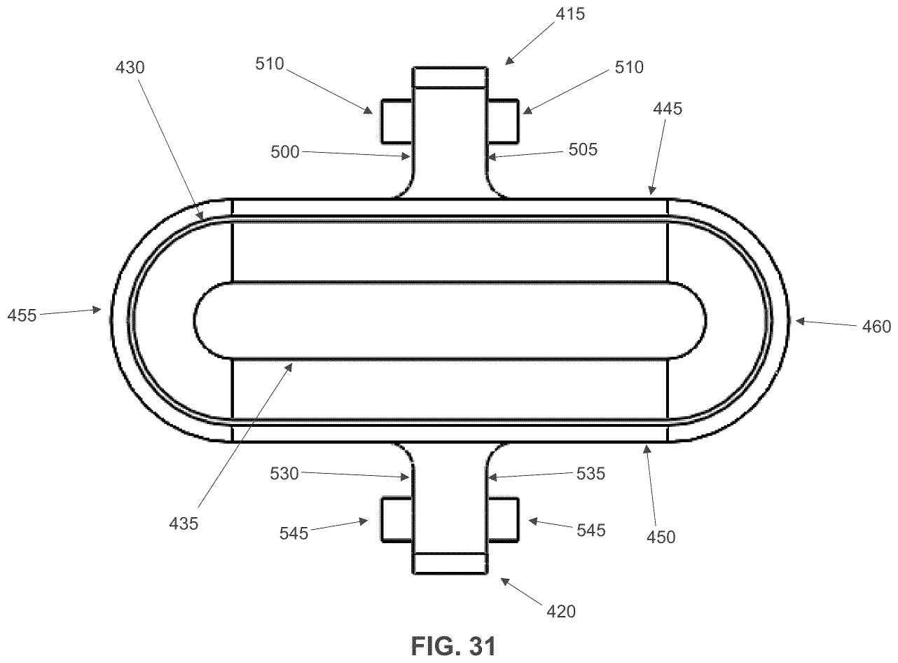

More particularly, and looking now at FIGS. 30 and 31, there is shown another alternative novel stabilization implant 405. Stabilization implant 405 generally comprises a body 410, a superior stabilizer 415 and an inferior stabilizer 420.

Body 410 generally comprises a rounded rectangular cross-section, an ovoid cross-section, a laterally-extending cross-section, or such other cross-section as will inhibit rotation of body 410 about its central longitudinal axis when stabilization implant 405 is disposed in sacroiliac joint 60, as will hereinafter be discussed in further detail. By way of example but not limitation, body 410 may comprise a circular cross-section, a square cross-section, a “star-shaped” cross-section, a triangular cross-section, etc.

More particularly, body 410 comprises a distal end 425 characterized by a circumferentially-extending distal end cutting surface 430 defining a distal mouth 435, a proximal end 440, a superior surface 445, an inferior surface 450, a first lateral surface 455 and a second lateral surface 460.

In one preferred form of the invention, a superior chamfer surface 465 extends between superior surface 445 and circumferentially-extending distal end cutting surface 430, an inferior chamfer surface 470 extends between inferior surface 450 and circumferentially-extending distal end cutting surface 430, a first lateral chamfer surface 475 extends between first lateral surface 455 and circumferentially-extending distal end cutting surface 430, and a second lateral chamfer surface 480 extends between second lateral surface 460 and circumferentially-extending distal end cutting surface 430. As a result of this construction, it will be appreciated that distal end 425 of stabilization implant 405 comprises a tapered distal end for facilitating insertion into cavity 45 formed in sacroiliac joint 60.

In one preferred form of the invention, first lateral surface 455 and second lateral surface 460 comprise curved surfaces, whereby to provide body 410 of stabilization implant 405 with a generally ovoid cross-section that matches the cross-section of the bone cavity in which stabilization implant 405 is to be inserted, as will hereinafter be discussed in further detail below.

If desired, body 410 may comprise a hollow central cavity 485 opening on distal mouth 435 via a distal opening 490 for facilitating bony in-growth into stabilization implant 405 and/or for carrying bone-promoting material. Cavity 485 may pass partly into body 410, or may pass entirely through body 410.

Superior stabilizer 415 generally comprises a first lateral stabilizer surface 500, a second lateral stabilizer surface 505, and an angled superior stabilizer surface 507. The distance between first lateral stabilizer surface 500 and second lateral stabilizer surface 505 (i.e., the width of superior stabilizer 415) comprises a width slightly smaller than, equal to, or just slightly larger than the gap between the opposing articular surfaces of the joint into which stabilizer implant 405 is to be inserted. By way of example but not limitation, where stabilizer implant 405 is to be disposed in a cavity 45 formed in sacroiliac joint 60, the distance between first lateral stabilizer surface 500 and second lateral stabilizer surface 505 preferably comprises a width just slightly larger than the gap between the opposing articular surfaces of the ilium and the sacrum such that superior stabilizer 415 can make a snug fit (e.g., a “press-fit”) therebetween.

In one preferred form of the present invention, a plurality of tabs 510 are formed on one or both of first lateral stabilizer surface 500 and second lateral stabilizer surface 505, whereby to grip the surface of articulating bones disposed on either side of superior stabilizer 415 when stabilization implant 405 is disposed in the gap between the opposing articular surfaces of a joint, as will be apparent to one of skill in the art in view of the present disclosure. Tabs 510 extend laterally outboard of one or both of first lateral stabilizer surface 500 and second lateral stabilizer surface 505. Each tab 510 preferably comprises an angled distal surface 515 for facilitating insertion of stabilization implant 405 into the joint space, and a proximal surface 520 disposed generally perpendicular to lateral stabilizer surfaces 500, 505 in order to prevent retrograde (i.e., proximal) movement of stabilization implant 405 out of the joint space after implantation. The lateral outboard distance to which tabs 510 extend may be varied, with some tabs 510 extending laterally outboard a first distance, and some tabs 510 extending laterally outboard a second distance which is greater than the first distance.

Inferior stabilizer 420 generally comprises a first lateral stabilizer surface 530, a second lateral stabilizer surface 535, and an angled inferior stabilizer surface 540. The distance between first lateral stabilizer surface 530 and second lateral stabilizer surface 535 (i.e., the width of inferior stabilizer 420) comprises a width slightly smaller than, equal to, or just slightly larger than the gap between the opposing articular surfaces of the joint into which stabilizer implant 405 is to be inserted. By way of example but not limitation, where stabilizer implant 405 is to be disposed in a cavity 45 formed in sacroiliac joint 60, the distance between first lateral stabilizer surface 530 and second lateral stabilizer surface 535 comprises a width just slightly larger than the gap between the opposing articular surfaces of the ilium and the sacrum such that inferior stabilizer 420 can make a snug fit (e.g., a “press-fit”) therebetween.

In one preferred form of the present invention, a plurality of tabs 545 are formed on one or both of first lateral stabilizer surface 530 and second lateral stabilizer surface 535, whereby to grip the surface of articulating bones disposed on either side of inferior stabilizer 420 when stabilization implant 405 is disposed in the gap between the opposing articular surfaces of a joint, as will be apparent to one of skill in the art in view of the present disclosure. Tabs 545 extend laterally outboard of one or both of first lateral stabilizer surface 530 and second lateral stabilizer surface 535. Each tab 545 preferably comprises an angled distal surface 550 for facilitating insertion of stabilization implant 405 into the joint space, and a proximal surface 555 disposed generally perpendicular to lateral stabilizer surfaces 530, 535 in order to prevent retrograde (i.e., proximal) movement of stabilization implant 405 out of the joint space after implantation. The lateral outboard distance to which tabs 545 extend may be varied, with some tabs 545 extending laterally outboard a first distance, and some tabs 545 extending laterally outboard a second distance which is greater than the first distance.

In use, stabilization implant 405 is inserted into a cavity formed in the articulating surfaces of two adjacent bones that define a joint space. By way of example but not limitation, where implant 405 is to be inserted into the sacroiliac joint, cavity 45 is formed using one of the surgical techniques discussed above, with cavity 45 being dimensioned so as to be sufficiently deep and proportionally distributed in sacrum 50 and ilium 55 to receive stabilization implant 405 therein. Implant 405 is then inserted into cavity 45 (e.g., using the novel technique discussed above) such that superior stabilizer 415 and inferior stabilizer 420 make a press-fit with articulating surfaces of sacrum 50 and ilium 55. It will be appreciated that superior stabilizer 415 and inferior stabilizer 420 engage the surrounding bone so as to inhibit rotation of body 410 within sacroiliac joint 60, and act to stabilize sacrum 50 relative to ilium 55 such that sacroiliac joint 60 is immobilized, whereby to facilitate joint fusion. It will also be appreciated that tabs 510 on first lateral stabilizer surface 500 and second lateral stabilizer surface 505 of superior stabilizer 415 and tabs 545 on first lateral stabilizer surface 530 and second lateral stabilizer surface 535 of inferior stabilizer 420 help to prevent stabilization implant 405 from “backing out” of sacroiliac joint 60 (i.e., retrograde movement) after stabilization implant 405 has been positioned in sacroiliac joint 60. It should also be appreciated that tabs 510, 545 engage the articulating surfaces of sacrum 50 and ilium 55 such that the articulating surfaces do not move relative to stabilization implant 405, thereby stabilizing sacroiliac joint 60 and promoting bone fusion across sacroiliac joint 60. And it should further be appreciated that the provision of distal mouth 435 and hollow central cavity 485 provide clear avenues for bony in-growth into (or through) stabilization implant 405 across sacroiliac joint 60, further enhancing stabilization of sacroiliac joint 60 (e.g., via bone fusion).

Although stabilization implant 405 has been disclosed above in the context of fusing a sacroiliac joint, it should also be appreciated that stabilization implant 405 may be used to stabilize and fuse any joint having anatomy similar to the sacroiliac joint, i.e., a pair of opposing bony surfaces defining a gap therebetween, with superior stabilizer 415 and inferior stabilizer 420 being sized to be positioned within the joint gap. By way of example but not limitation, stabilization implant 405 may also be used in small joints such as the fingers, toes, etc.

### Surgical Technique for Implanting Stabilization Implants 205, 405 in Sacroiliac Joint 60

As stated above, stabilization implants 205 and 405 are preferably implanted into sacroiliac joint 60 in the same manner as is utilized to implant the aforementioned stabilization implant 5 into sacroiliac joint 60 (see the above sections entitled “Detailed Surgical Technique” and “Alternative Surgical Technique”).

Although stabilization of sacroiliac joint 60 may be achieved by implanting a single stabilization implant 205 (and/or a single stabilization implant 405) into an sacroiliac joint 60, it should be appreciated that the method of stabilizing sacroiliac joint 60 of the present invention is not limited to the use of a single stabilization implant 205, 405 in a single sacroiliac joint 60. The present invention also encompasses implanting a plurality of stabilization implants 205, 405 in a sacroiliac joint 60 and/or implanting a plurality of stabilization implants 205, 405 into both sacroiliac joints (i.e., the current sacroiliac joint 60 and the contralateral sacroiliac joint 60). Moreover, if desired, a combination of one or more stabilization implants 5 may be implanted in a sacroiliac joint 60.

### MODIFICATIONS

It should be understood that many additional changes in the details, materials, steps and arrangements of parts, which have been herein described and illustrated in order to explain the nature of the present invention may be made by those skilled in the art while still remaining within the principles and scope of the invention.

It will be seen that the advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

What is claimed is:

1. A stabilization implant comprising:
an elongated body having a distal end, a proximal end and a longitudinal axis extending between the distal end and the proximal end, the elongated body being characterized by a superior body surface and an inferior body surface;
a superior stabilizer extending outwardly from the superior body surface, the superior stabilizer being characterized by a first lateral superior stabilizer surface and a second lateral superior stabilizer surface; and
an inferior stabilizer extending outwardly from the inferior body surface, the inferior stabilizer being characterized by a first lateral inferior stabilizer surface and a second lateral inferior stabilizer surface;
wherein at least one of the first lateral superior stabilizer surface, the second lateral superior stabilizer surface, the first lateral inferior stabilizer surface, and the second lateral inferior stabilizer surface comprise at least one outwardly-extending tab.

2. A stabilization implant according to claim 1 wherein the at least one outwardly-extending tab comprises an angled distal surface and a proximal surface which is disposed generally perpendicular to the first lateral superior stabilizer surface, the second lateral superior stabilizer surface, the first lateral inferior stabilizer surface, and/or the second lateral inferior stabilizer surface out of which the at least one outwardly-extending tab extends.

3. A stabilization implant according to claim 1 wherein the first lateral superior stabilizer surface, the second lateral superior stabilizer surface, the first lateral inferior stabilizer surface, and the second lateral inferior stabilizer surface comprise at least two outwardly-extending tabs.

4. A stabilization implant according to claim 3 wherein a first tab of the at least two outwardly-extending tabs extends outward a first distance, a second tab of the at least two outwardly-extending tabs extends outward a second distance, and further wherein the first distance is greater than the second distance.

5. A stabilization implant according to claim 1 wherein the elongated body comprises at least one window formed in the elongated body for promoting bony in-growth.

6. A stabilization implant according to claim 5 wherein the at least one window comprises a bone growth promoter.

7. A stabilization implant according to claim 5 wherein the at least one window passes through the elongated body so as to open on the superior body surface and the inferior body surface.

8. A stabilization implant according to claim 5 wherein the at least one window passes through the elongated body so as to open on the first lateral surface and the second lateral surface.

9. A stabilization implant according to claim 1 wherein the distal end of the elongated body terminates in a distal mouth, and further wherein the distal mouth extends proximally into the elongated body.

10. A stabilization implant according to claim 9 wherein the distal mouth comprises a distalmost circumferentially-extending distal end cutting surface.

11. A stabilization implant according to claim 1 wherein the stabilization implant is asymmetric about the longitudinal axis of the elongated body.

12. A stabilization implant according to claim 1 wherein the elongated body is symmetrical about the longitudinal axis of the elongated body.

13. A stabilization implant according to claim 1 wherein the superior stabilizer and the inferior stabilizer have differing profiles in side view.

14. A stabilization implant according to claim 1 wherein the distal end of the elongated body terminates in a distal end surface, wherein the elongated body comprises a beveled surface extending between the superior body surface and the distal end surface, and wherein the elongated body comprises a beveled surface extending between the inferior body surface and the distal end surface.

15. A stabilization implant according to claim 1 wherein the beveled surface extending between the superior body surface and the distal end surface extends at a first angle to the longitudinal axis of the elongated body, and the beveled surface extending between the inferior body surface and the distal end surface extends at a second angle to the longitudinal axis of the elongated body, and further wherein the first angle and the second angle are the same.

16. A stabilization implant according to claim 1 wherein the elongated body comprises a proximal end surface, the superior stabilizer comprises a proximal end surface and the inferior stabilizer comprises a proximal end surface, and further wherein the proximal end surface of the elongated body, the proximal end surface of the superior stabilizer and the proximal end surface of the inferior stabilizer are all coplanar.

17. A method of positioning an implant in a sacroiliac joint, the method comprising:
providing a stabilization implant comprising:
an elongated body having a distal end, a proximal end and a longitudinal axis extending between the distal end and the proximal end, the elongated body being characterized by a superior body surface and an inferior body surface;
a superior stabilizer extending outwardly from the superior body surface, the superior stabilizer being characterized by a first lateral superior stabilizer surface and a second lateral superior stabilizer surface; and
an inferior stabilizer extending outwardly from the inferior body surface, the inferior stabilizer being characterized by a first lateral inferior stabilizer surface and a second lateral inferior stabilizer surface;
wherein at least one of the first lateral superior stabilizer surface, the second lateral superior stabilizer surface, the first lateral inferior stabilizer surface, and the second lateral inferior stabilizer surface comprise at least one outwardly-extending tab;
forming an implant cavity for receiving the elongated body of the stabilization implant, wherein forming the implant cavity comprises forming a first cavity in an ilium of the sacroiliac joint and forming a second cavity in a sacrum of the sacroiliac joint, with the implant cavity spanning the sacroiliac joint; and
inserting the elongated body of the stabilization implant into the implant cavity.

18. A stabilization implant comprising:
an elongated body having a distal end, a proximal end and a central lumen extending between the distal end and the proximal end;
wherein the distal end of the elongated body terminates in a circumferentially-extending distal end cutting surface.

19. A stabilization implant according to claim 18 wherein the elongated body has a cross-section selected from the group consisting of a circular cross-section, an ovoid cross-section, a rectangular cross-section, a square cross-section, a “star-shaped” cross-section and a triangular cross-section.

20. A method of positioning an implant in a sacroiliac joint, the method comprising:
providing a stabilization implant comprising:
an elongated body having a distal end, a proximal end and a central lumen extending between the distal end and the proximal end;
wherein the distal end of the elongated body terminates in a circumferentially-extending distal end cutting surface; and
inserting the elongated body of the stabilization implant into the sacroiliac joint, with a first portion of the stabilization implant disposed in an ilium of the sacroiliac joint and a second portion of the stabilization implant disposed in a sacrum of the sacroiliac joint.
